(19) United States
(12) Patent Application Publication
(10) Pub. No.: US 2022/0071703 A1
(43) Pub. Date: Mar. 10, 2022

(54) SYSTEMS FOR MONITORING ABLATION PROGRESS USING REMOTE TEMPERATURE PROBES

(71) Applicant: Covidien LP, Mansfield, MA (US)

(72) Inventor: Jeetendra S. Bharadwaj, Erie, CO (US)

(21) Appl. No.: 17/419,424

(22) PCT Filed: Jan. 7, 2020
(86) PCT No.: PCT/US20/12444

§ 371 Date: Jun. 29, 2021

Related U.S. Application Data
(60) Provisional application No. 62/789,007, filed on Jan. 7, 2019.

(51) Int. Cl. A61B 18/18; A61B 34/20; A61B 90/00; G16H 20/40; G16H 40/63

(52) U.S. Cl. CPC A61B 18/1815; A61B 34/20; A61B 90/37; A61B 2018/00791; G16H 40/63; A61B 2034/2051; A61B 2090/378; G16H 20/40

(57) ABSTRACT

Disclosed are systems and methods for performing a microwave ablation procedure. An illustrative electrosurgical system includes a temperature probe including a temperature sensor, an ultrasound transducer, an electromagnetic (EM) tracking system including an EM field generator and an EM sensor coupled to the temperature probe, a display device, and a computing device including a processor and a memory storing instructions which, when executed by the processor, cause the computing device to receive, from the EM tracking system, tracking data indicating a position of the EM sensor, receive, from the ultrasound transducer, ultrasound images, determine a position of the temperature probe based on the position of the EM sensor, determine a trajectory of the temperature probe based on the position of the EM sensor, and cause the display device to display an indication of the position and the trajectory of the temperature probe on the ultrasound images.

### CROSS-REFERENCE TO RELATED APPLICATIONS

This application claims the benefit of the filing date of provisional U.S. Patent Application No. 62/789,007, filed Jan. 7, 2019.

### FIELD

The present disclosure relates to systems and methods for planning for and performing a microwave ablation treatment procedure. More specifically, the present disclosure describes using a remote temperature probe to monitor and/or control application of microwave energy to tissue in a treatment zone.

### BACKGROUND

Accurate placement of surgical tools prior to and during performance of surgical procedures is critical to ensuring successful performance of the surgical procedures. During traditional open surgical procedures, surgeons are able to at least partially see inside a patient's body to place the surgical tools at a treatment site and to determine progress and/or extent of a surgical procedure. However, during minimally-invasive surgical procedures, such as percutaneous surgical procedures, the surgeons are not able to see inside the patient's body, and thus alternative methods of visualization are needed to confirm accurate placement of the surgical tools and determine progress and/or extent of the surgical procedure.

Various methods for visualization of treatment sites inside a patient's body have been developed. For example, radiographic and/or ultrasound imaging may be utilized to confirm placement of surgical tools during minimally-invasive surgical procedures. However, improvements are needed in systems and methods for determining progress and/or extent of minimally-invasive surgical procedures.

### SUMMARY

Systems and methods for planning and performing a microwave ablation treatment procedure are provided.

Disclosed in accordance with embodiments of the present disclosure is an electrosurgical system including a temperature probe including a temperature sensor, an ultrasound transducer, an electromagnetic (EM) tracking system including an EM field generator and an EM sensor coupled to the temperature probe, a display device, and a computing device including a processor and a memory storing instructions which, when executed by the processor, cause the computing device to receive, from the EM tracking system, tracking data indicating a position of the EM sensor, receive, from the ultrasound transducer, ultrasound images, determine a position of the temperature probe based on the position of the EM sensor, determine a trajectory of the temperature probe based on the position of the EM sensor, and cause the display device to display an indication of the position and the trajectory of the temperature probe on the ultrasound images.

In another aspect of the present disclosure, the system further includes a microwave energy source and a microwave ablation antenna operably coupled to the microwave energy source, and the EM tracking system further includes a second EM sensor coupled to the microwave ablation antenna.

In a further aspect of the present disclosure, the instructions, when executed by the processor, further cause the computing device to receive, from the EM tracking system, tracking data indicating a position of the second EM sensor, determine a position of the microwave ablation antenna based on the position of the second EM sensor, determine a trajectory of the microwave ablation antenna based on the position of the second EM sensor, and cause the display device to display an indication of the position and the trajectory of the microwave ablation antenna on the ultrasound images.

In yet a further aspect of the present disclosure, the instructions, when executed by the processor, further cause the computing device to receive ablation procedure configuration settings, determine a projected ablation zone based on the ablation procedure configuration settings, determine a position of the projected ablation zone based on the position of the microwave ablation antenna, and cause the display device to display an indication of the position of the projected ablation zone on the ultrasound images.

In another aspect of the present disclosure, the instructions, when executed by the processor, further cause the computing device to receive a temperature measurement from the temperature probe, and determine whether the temperature measurement is above a threshold.

In a further aspect of the present disclosure, the instructions, when executed by the processor, further cause the computing device to, when it is determined that the temperature measurement is above the threshold, cause the microwave energy source to stop providing microwave energy to the microwave ablation antenna.

In another aspect of the present disclosure, the instructions, when executed by the processor, further cause the computing device to, when it is determined that the temperature measurement is above the threshold, cause the display device to display an indication that the temperature measurement is above the threshold.

In yet another aspect of the present disclosure, the instructions, when executed by the processor, further cause the computing device to receive ablation procedure configuration settings, and the threshold is determined based on the ablation procedure configuration settings.

In still another aspect of the present disclosure, the threshold is a predetermined value.

In yet another aspect of the present disclosure, the instructions, when executed by the processor, further cause the computing device to receive a temperature measurement from the temperature probe determine a progress of an ablation procedure based on the temperature measurement.

In a further aspect of the present disclosure, the instructions, when executed by the processor, further cause the computing device to cause the display device to display an indication of the progress of the ablation procedure.

In another aspect of the present disclosure, the instructions, when executed by the processor, further cause the computing device to determine, based on the progress of the ablation procedure, whether the ablation procedure has been completed.

In a further aspect of the present disclosure, the instructions, when executed by the processor, further cause the computing device to, when it is determined that the ablation procedure has been completed, cause the microwave energy source to stop providing microwave energy to the microwave ablation antenna.

In another aspect of the present disclosure, the instructions, when executed by the processor, further cause the computing device to, when it is determined that the ablation procedure has been completed, cause the display device to display an indication that the ablation procedure has been completed.

In yet another aspect of the present disclosure, the instructions, when executed by the processor, further cause the computing device to receive a temperature measurement from the temperature probe, and cause the display device to display an indication of the temperature measurement.

In still another aspect of the present disclosure, the EM tracking system further includes a second EM sensor coupled to the ultrasound transducer, and wherein the instructions, when executed by the processor, further cause the computing device to determine a position of an ultrasound image plane generated by the ultrasound transducer based on the position of the second EM sensor, determine a point of intersection between the trajectory of the temperature probe and the ultrasound image plane, and cause the display device to display an indication of the point of intersection between the trajectory of the temperature probe and the ultrasound image plane.

In yet another aspect of the present disclosure, the EM tracking system further includes a second EM sensor coupled to the ultrasound transducer, and wherein the instructions, when executed by the processor, further cause the computing device to determine a position of an ultrasound image plane generated by the ultrasound transducer, based on the position of the second EM sensor, determine a point of intersection between the temperature probe and the ultrasound image plane, and cause the display device to display an indication of the point of intersection between the temperature probe and the ultrasound image plane.

In still another aspect of the present disclosure, the tracking data is first tracking data and the position of the EM sensor is a first position of the EM sensor, and wherein the instructions, when executed by the processor, further cause the computing device to receive, from the EM tracking system, second tracking data indicating a second position of the EM sensor, determine an updated position of the temperature probe based on the second position of the EM sensor, determine an updated trajectory of the temperature probe based on the second position of the EM sensor, and cause the display device to display an indication of the updated position and the updated trajectory of the temperature probe on the ultrasound images.

Disclosed in accordance with embodiments of the present disclosure is a method including receiving tracking data indicating a position of an EM sensor coupled to a temperature probe, receiving, from an ultrasound transducer, ultrasound images, determining a position of the temperature probe based on the position of the EM sensor, determining a trajectory of the temperature probe based on the position of the EM sensor, and causing a display device to display an indication of the position and the trajectory of the temperature probe on the ultrasound images.

Disclosed in accordance with embodiments of the present disclosure is a non-transitory computer-readable medium storing instructions which, when executed by a processor, cause a computing device to receive tracking data indicating a position of an EM sensor coupled to a temperature probe, receive, from an ultrasound transducer, ultrasound images, determine a position of the temperature probe based on the position of the EM sensor, determine a trajectory of the temperature probe based on the position of the EM sensor, and cause a display device to display an indication of the position and the trajectory of the temperature probe on the ultrasound images.

Any of the above aspects and embodiments of the present disclosure may be combined without departing from the scope of the present disclosure.

### BRIEF DESCRIPTION OF THE DRAWINGS

Objects and features of the presently disclosed system and method will become apparent to those of ordinary skill in the art when descriptions of various embodiments thereof are read with reference to the accompanying drawings, of which:

FIG. 1 is a schematic diagram of a microwave ablation planning and procedure system in accordance with an illustrative embodiment of the present disclosure;

FIG. 2 is a schematic diagram of a computing device which forms part of the microwave ablation planning and procedure system of FIG. 1 in accordance with an embodiment of the present disclosure;

FIGS. 3A-E show a flowchart illustrating an example method of using a remote temperature probe to monitor progress of a microwave ablation treatment, in accordance with an embodiment of the present disclosure; and

FIG. 4 is an illustration of a graphical user interface that may be displayed by the computing device of FIG. 2, in accordance with an embodiment of the present disclosure.

### DETAILED DESCRIPTION

The present disclosure provides systems and methods for using one or more remote temperature probes during microwave ablation treatment to monitor progress and extent of the microwave ablation treatment. The present disclosure further provides systems and methods for assisting a clinician in placement of tools, such as remote temperature probes and microwave ablation probes, during minimally-invasive surgical procedures. The remote temperature probes may be positioned about a treatment location to monitor a change in temperature of tissue as microwave ablation energy is applied to the tissue, and the system may suspend the microwave ablation procedure once a predetermined temperature is reached. Additionally, remote temperature probes may be positioned proximate critical structures that are within or adjacent the treatment location to monitor a change in temperature at locations of such critical structures to determine when the microwave ablation procedure should be suspended to prevent injury to the critical structures.

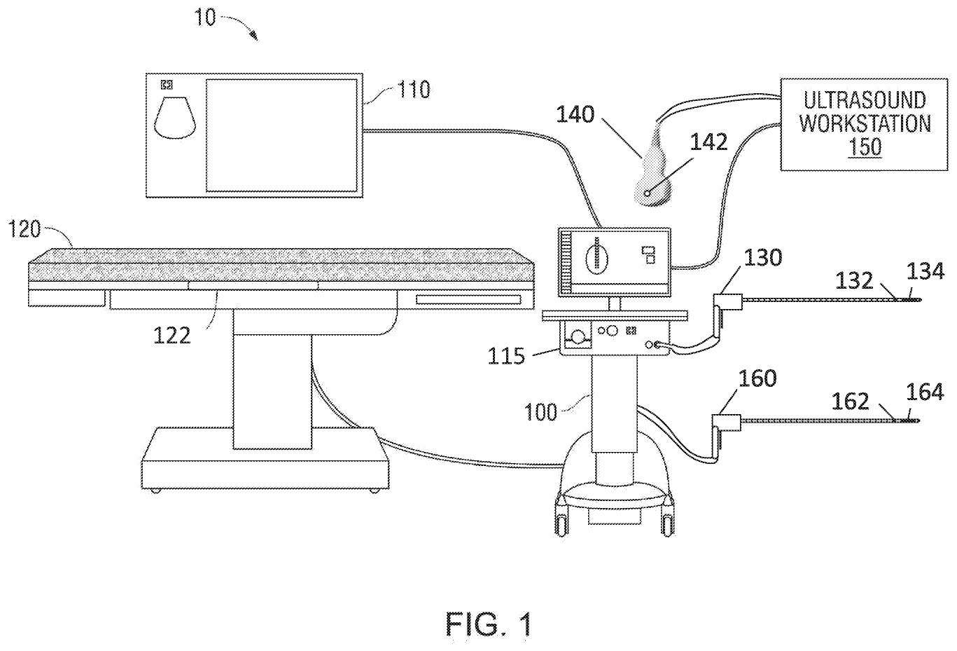

Referring now to FIG. 1, the present disclosure is generally directed to a treatment system 10, which includes a computing device 100, a display 110, a table 120, an ablation probe 130, an ultrasound sensor 140, an ultrasound workstation 150, and a remote temperature probe (RTP) 160. The computing device 100 may be, for example, a laptop computer, a desktop computer, a tablet computer, and/or other similar device. The computing device 100 may be configured to control and/or receive data from an electrosurgical generator 115, a peristaltic pump (not expressly shown in FIG. 1), a power supply (not expressly shown in FIG. 1), and/or any other accessories and peripheral devices relating to, or forming part of, the system 10. The computing device 100 further controls and/or receives data from the ultrasound workstation 150 and the RTP 160.

The display 110 is configured to output instructions, images, and messages relating to the microwave ablation procedure. The computing device 100 may also include a display that may be configured to output instructions, images, and/or messages relating to the microwave ablation procedure. The table 120 may be, for example, an operating table or other table suitable for use during a surgical procedure. The table 120 includes an electromagnetic (EM) field generator 122 that is used to generate an EM field during the microwave ablation procedure. The EM field generator 122 forms part of an EM tracking system used to track the positions of instruments, such as the ablation probe 130, the ultrasound sensor 140, and/or the RTP 160, within the EM field. The EM field generator 122 may include various components, such as a specially designed pad to be placed under, or integrated into, an operating table or patient bed. An example of such an EM tracking system is the AURORA system sold by Northern Digital Inc. The EM tracking system further includes various EM sensors 132, 142, 162 coupled to or included in instruments, as described further below. The EM tracking system provides data regarding the EM field and the tracked positions of the EM sensors 132, 142, 162 to the computing device 100. The computing device 100 uses the data received from the EM tracking system to determine positions of the instruments relative to each other and to marked objects, as further described below.

The ablation probe 130 is a surgical instrument having a microwave ablation antenna which is used to ablate tissue. The ablation probe 130 receives microwave energy from the generator 115. The ablation probe 130 includes an EM sensor 132 by means of which the EM tracking system tracks the position of the ablation probe 130. An example method of tracking the location of the ablation probe 130 includes using the EM tracking system, which tracks the location of the ablation probe 130 by tracking the EM sensor 132 coupled to or incorporated within the ablation probe 130. Various types of sensors may be used, such as a printed sensor, the construction and use of which is more fully described in U.S. Patent Appl. Publ. No. 2016/0174873, entitled MEDICAL INSTRUMENT WITH SENSOR FOR USE IN A SYSTEM AND METHOD FOR ELECTROMAGNETIC NAVIGATION, filed Oct. 22, 2015, by Greenburg et al. the entire contents of which is incorporated herein by reference. The ablation probe 130 further includes a radiating portion 134 where microwave energy is emitted when the ablation probe 130 is activated. The ablation probe 130 is used to ablate a lesion or tumor (hereinafter referred to as a “target”) by using electromagnetic radiation or microwave energy to heat tissue in order to denature or kill cancerous cells. The construction and use of a system including such an ablation probe 130 is more fully described in U.S. Patent Appl. Publ. No. 2016/0058507, entitled MICROWAVE ABLATION SYSTEM, filed on Aug. 18, 2015, by William J. Dickhans, U.S. Pat. No. 9,247,992, entitled MICROWAVE ABLATION CATHETER AND METHOD OF UTILIZING THE SAME, filed on Mar. 15, 2013, by Ladtkow et al., and U.S. Pat. No. 9,119,650, entitled MICROWAVE ENERGY-DELIVERY DEVICE AND SYSTEM, filed on Mar. 15, 2013, by Brannan et al., the contents of each of which are hereby incorporated by reference in its entirety.

The ultrasound sensor 140 may be any ultrasound device which, in conjunction with the ultrasound workstation 150, provides ultrasound data and/or images to the computing device 100. In embodiments, the ultrasound sensor 140, such as an ultrasound wand or transducer, may be used to image the patient's body during the microwave ablation procedure to visualize the location of the surgical instruments, such as the ablation probe 130 and/or the RTP 160, and/or structures or objects inside the patient's body. The ultrasound sensor 140 has an EM tracking sensor 142 included therein or coupled thereto, for example, a clip-on sensor or a sticker sensor. As described further below, the ultrasound sensor 140 may be positioned in relation to the ablation probe 130 and/or the RTP 160 such that the ablation probe 130 or the RTP 160 is at an angle to the ultrasound image plane, thereby enabling the clinician to visualize the spatial relationship of the ablation probe 130 and/or the RTP 160 with the ultrasound image plane and with the objects being imaged. The EM tracking system may also track the position of the ultrasound sensor 140 within the EM field to enable the computing device 100 to determine a location of the ultrasound image plane relative to marked objects within the EM field, as described further below. In some embodiments, one or more ultrasound sensors 140 may be placed inside the body of the patient. The EM tracking system may then track the location of such ultrasound sensors 140, the ablation probe 130, and/or the RTP 160 inside the body of the patient.

The RTP 160 may be any surgical device that includes an EM sensor 162 and a temperature sensor 164. For the purpose of clarity, in the embodiments described below, RTP 160 is described as a surgical instrument dedicated to the purpose of monitoring temperature. However, those skilled in the art will appreciate that RTP 160 may form part of another surgical instrument, such as a second ablation probe, a vessel sealing device, a surgical stapler, etc., and is used to monitor temperature during the microwave ablation procedure prior to or after performing another function.

While the present disclosure describes the use of system 10 in a surgical environment, it is also envisioned that some or all of the components of system 10 may be used in alternative settings, for example, an imaging laboratory and/or an office setting. Additionally, those skilled in the art will appreciate that various other surgical instruments or tools, such as vessel sealing devices, surgical staplers, etc., may also be equipped with an EM sensor and used during the performance of a microwave ablation treatment procedure.

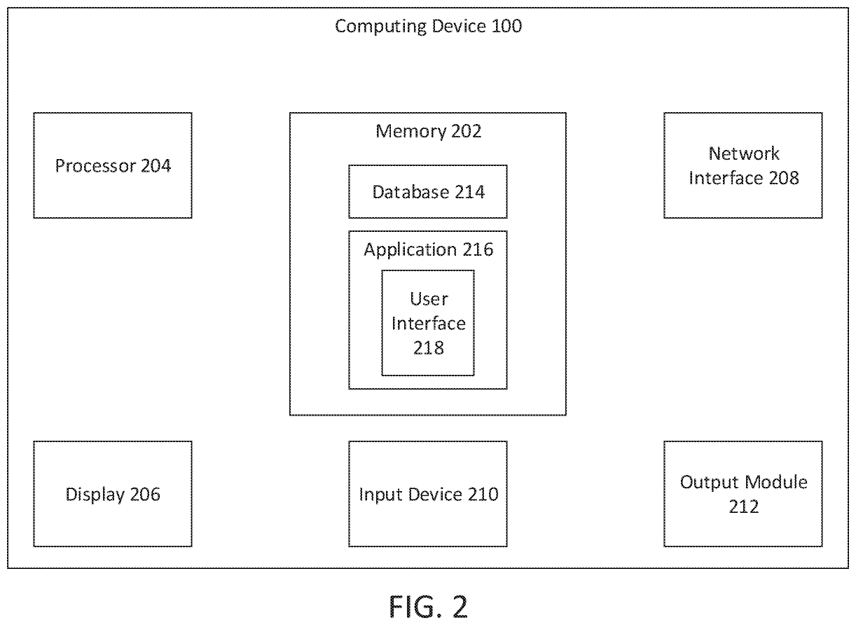

Turning now to FIG. 2, there is shown a schematic diagram of the computing device 100. The computing device 100 may include a memory 202, one or more processors 204, a display 206, a network interface 208, an input device 210, and/or an output module 212.

The memory 202 includes any non-transitory computer-readable storage media for storing data and/or software that is executable by the processors 204 and which controls the operation of the computing device 100. In an embodiment, the memory 202 may include one or more solid-state storage devices such as flash memory chips. Alternatively or in addition to the one or more solid-state storage devices, the memory 202 may include one or more mass storage devices connected to the processors 204 through a mass storage controller (not shown) and a communications bus (not shown). Although the description of computer-readable media included herein refers to a solid-state storage, it will be appreciated by those skilled in the art that computer-readable storage media may be any available media that can be accessed by the processors 204. That is, computer readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which can be used to store the desired information and which can be accessed by the computing device 100.

The memory 202 may store an application 216 and/or a database 214, which may in turn store various information and/or image data relating to the microwave ablation procedure, the surgical instruments, and/or the patient. The application 216 may include, among other things, instructions, such as computer-executable code, for displaying a graphical user interface (GUI) 218.

The processors 204 may include general purpose processors, specialized graphics processing units (GPU) configured to perform specific graphics processing tasks while freeing up the general purpose processors to perform other tasks, and/or any number or combination of such processors. The display 206 may be touch sensitive, voice activated, and/or controllable via computer-vision, enabling the display 206 to serve as both an input and output device. Alternatively, a keyboard (not shown), a mouse (not shown), and/or other data input devices may be employed.

The network interface 208 may be configured to connect to a network such as a local area network (LAN) consisting of a wired network and/or a wireless network, a wide area network (WAN), a wireless mobile network, a BLUETOOTH network, and/or the internet. For example, computing device 100 may receive ultrasound or radiographic image data of a patient from a server, for example, a hospital server, internet server, or other similar servers, for use during the microwave ablation procedure. Data may also be provided to the computing device 100 via a removable memory. The computing device 100 may receive updates to its software, for example, the application 216, via the network interface 208. The input device 210 may be any device by means of which a user may interact with computing device 100, such as, for example, a mouse, a keyboard, a foot pedal, a touch screen, and/or a voice and/or computer-vision interface. The output module 212 may include any connectivity port or bus, such as, for example, parallel ports, serial ports, universal serial busses (USB), or any other similar connectivity port known to those skilled in the art.

The application 216 may be one or more software programs stored in the memory 202 and executed by processors 204 of the computing device 100. As will be described in more detail below, during a planning or configuration phase of the microwave ablation procedure, the application 216 may guide a clinician through a series of steps to identify a target, size the target, size a treatment zone, and/or determine an access route to the target for use during the microwave ablation procedure. In some embodiments, the application 216 is loaded on computing devices in an operating room or other facility where surgical procedures are performed, and is used as a plan or map to guide a clinician performing a surgical procedure, but without any feedback from the ablation probe 130 and/or RTP 160 used in the procedure to indicate where ablation probe 130 and/or RTP 160 are located in relation to the plan. In other embodiments, the system 10 provides the computing device 100 with data regarding the location of the ablation probe 130 and/or the RTP 160 within the patient's body, such as by using the EM tracking system, which the application 216 then uses to indicate on the plan where the ablation probe 130 and/or RTP 160 are located.

The application 216 may be installed directly on the computing device 100, or may be installed on another computer, for example a central server, and opened on computing device 100 via the network interface 208. The application 216 may run natively on the computing device 100, as a web-based application, or any other format known to those skilled in the art. In some embodiments, the application 216 will be a single software program having all of the features and functionality described in the present disclosure. In other embodiments, the application 216 may be two or more distinct software programs providing various parts of these features and functionality. For example, the application 216 may include one software program for use during the planning phase, and a second software program for use during the procedure phase of the microwave ablation procedure. In such instances, the various software programs forming part of the application 216 may be enabled to communicate with each other and/or import and export various settings and parameters relating to the microwave ablation procedure and/or the patient to share information. For example, a treatment plan and any of its components generated by one software program during the planning phase may be stored and exported to be used by a second software program during the procedure phase.

The application 216 generates the 218 to present visual interactive features to a clinician, for example, on the display 206 and/or the display 110, and for receiving input from the clinician, for example, via the user input device 210. The computing device 100 is linked to the display 110, thus enabling the computing device 100 to control the output on the display 110 along with the output on the display 206. The computing device 100 may control the display 110 to display output which is the same as or similar to the output displayed on the display 206. For example, the output on the display 206 may be mirrored on the display 100. Alternatively, the computing device 100 may control the display 110 to display different output from that displayed on the display 206. For example, the display 110 may be controlled to display guidance images and information during the microwave ablation procedure, while the display 206 is controlled to display other output, such as configuration or status information.

As used herein, the term “clinician” refers to any medical professional (i.e., doctor, surgeon, nurse, technician, and/or the like) or other user of the system 10 involved in planning, performing, monitoring and/or supervising the microwave ablation procedure involving the use of the embodiments described herein.

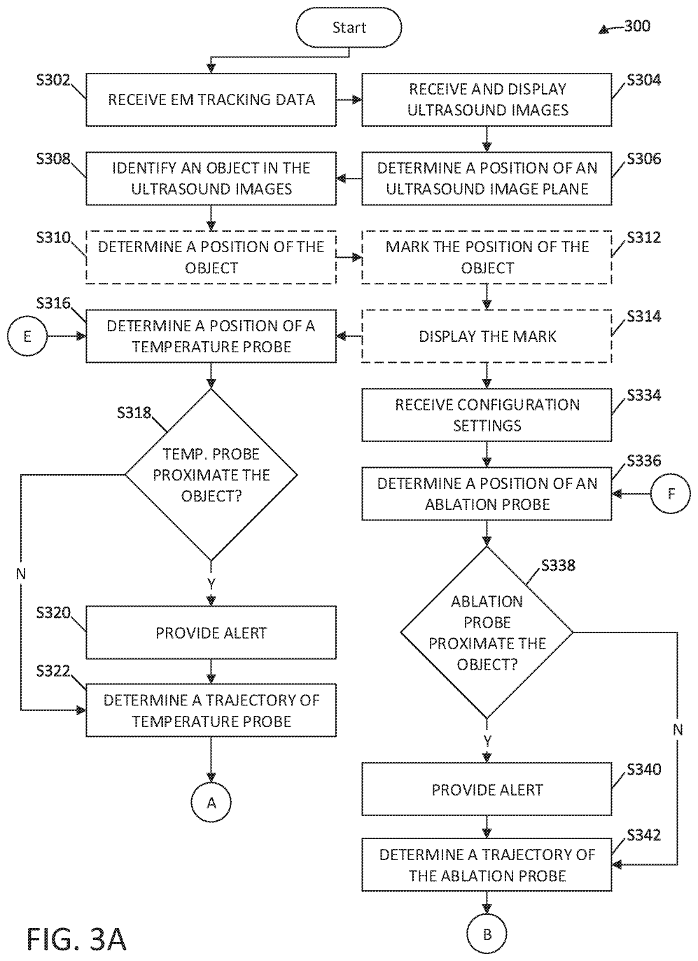

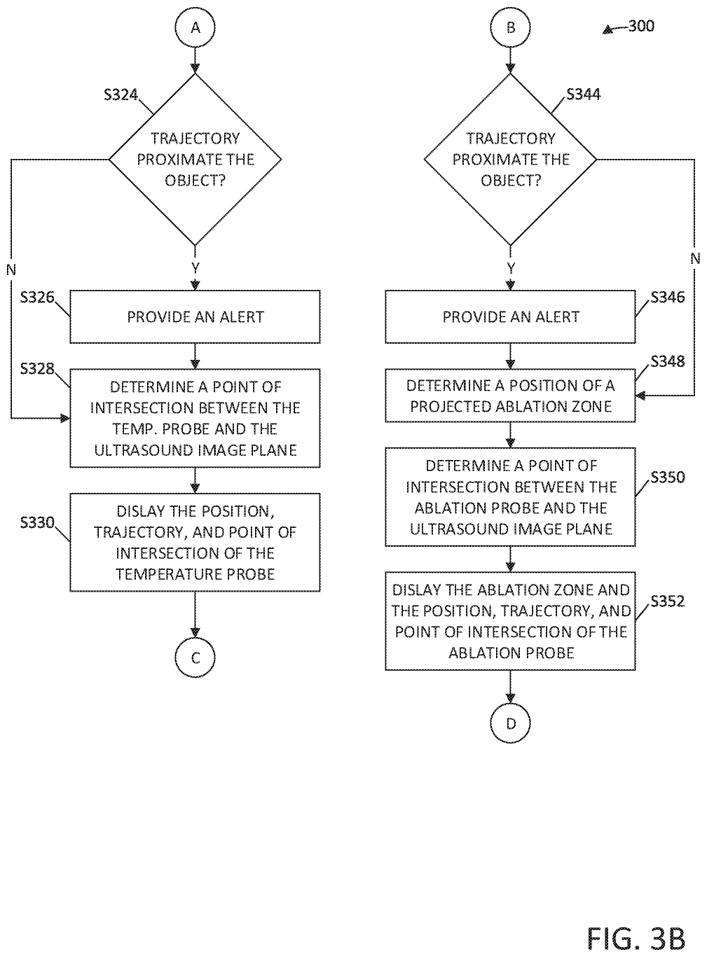

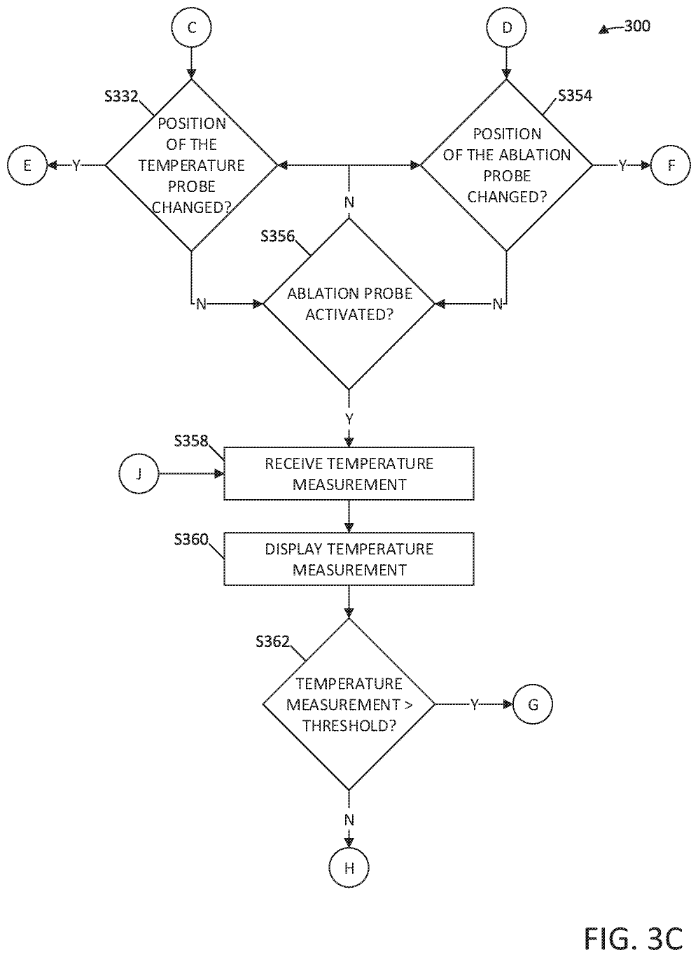

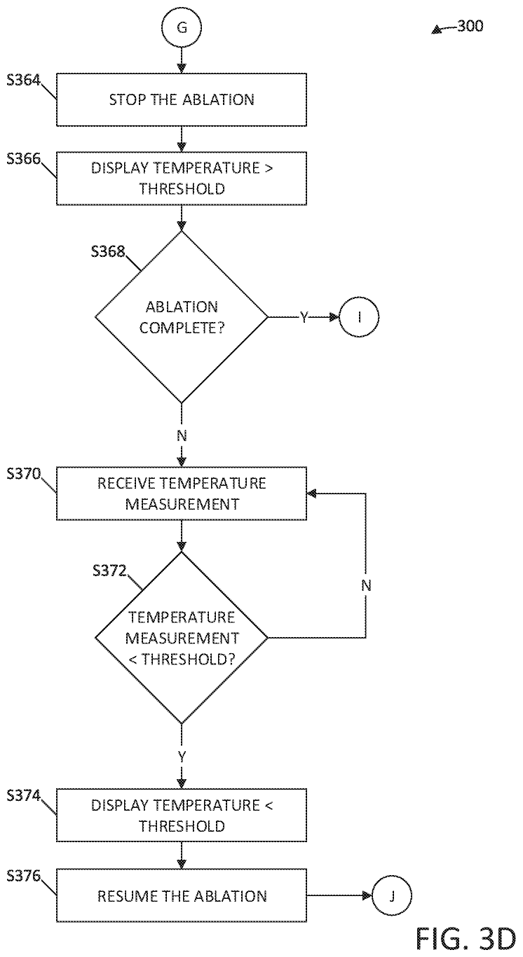

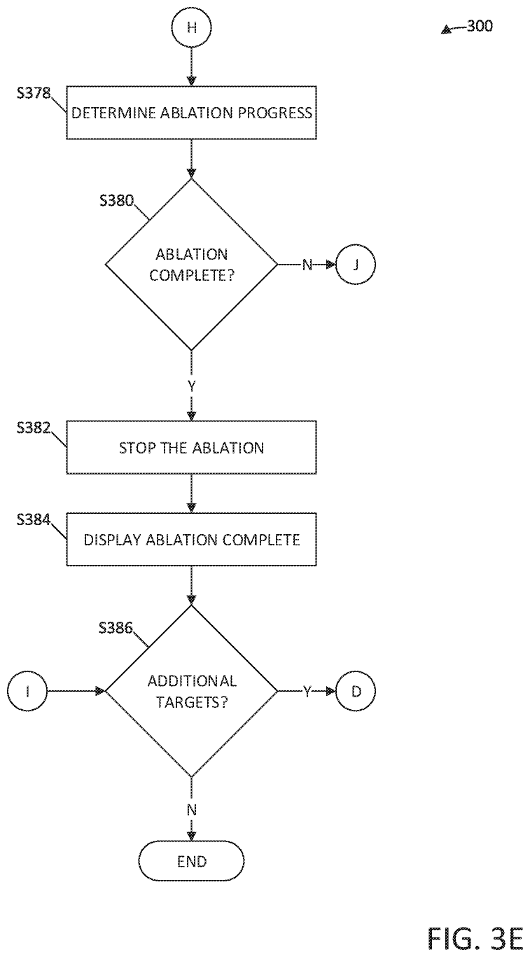

Turning now to FIGS. 3A-3E (referred to collectively herein as FIG. 3), there is shown a flowchart of an illustrative method 300 for performing a microwave ablation procedure, according to embodiments of the present disclosure. In some embodiments, a planning and/or configuration phase may precede the microwave ablation procedure described below with reference to FIG. 3. During such a planning and/or configuration phase, a clinician may use the computing device 100 to load a treatment plan into the application 216. The treatment plan may include radiographic images of the patient's body and/or a computer-generated model of the patient's body. The treatment plan may further include one or more previously-selected targets and predetermined access paths to the targets. In some embodiments, the clinician may further select one or more targets and/or identify access paths to the targets once the treatment plan is loaded. Configuration settings regarding the microwave ablation procedure, such as power, temperature, and/or duration of the microwave ablation, may also be loaded or entered during the planning and/or configuration phase. Further details regarding the planning and/or configuration phase can be found in U.S. Patent Appl. Publ. No. 2016/0317229, entitled METHODS FOR MICROWAVE ABLATION PLANNING AND PROCEDURE, filed on Apr. 15, 2016 by Girotto et al., the entire contents of which are incorporated herein by reference.

In other embodiments, the planning and/or configuration phase is not performed prior to the start of the microwave ablation procedure, and some of these functions are performed as part of the microwave ablation procedure, as described below in the description of the method 300. The method 300 may start at step S302 of FIG. 3A, where the computing device 100 receives EM tracking data from the EM tracking system. The EM tracking data may include data regarding the EM field generated by the EM field generator 122, as well as the positions of each of the EM sensors 132, 142, 162 detected in the EM field.

Thereafter, at step S304, the computing device 100 receives ultrasound images from the ultrasound sensor 140 via the ultrasound workstation 150. The computing device 100 may then cause the received ultrasound images to be displayed on one or both of the display 110 and/or the display 206. Next, at step S306, the computing device 100 determines a position of the ultrasound image plane and/or the ultrasound images within the EM field. For example, the computing device 100 may use the EM tracking data of EM sensor 142 received at step S302 to determine a position and orientation (referred to hereinafter as a “pose”) of the ultrasound sensor 140 within the EM field, and may determine, based on the pose of the ultrasound sensor 140, the position of the ultrasound image plane within the EM field. The computing device 100 may further determine, based on data regarding a scanning depth of the ultrasound sensor, a position of the ultrasound images. The ultrasound images will be in the ultrasound image plane, offset from the position of the ultrasound sensor 140 by the scanning depth of the ultrasound sensor 140.

Thereafter, or concurrently therewith, at step S308, an object is identified in the ultrasound images received at step S304. In some embodiments, a clinician may view the ultrasound images as displayed at step S304, visually identify an object in the ultrasound images, and provide input, such as via input device 210, to the computing device 100 to select the identified object. In other embodiments, the computing device 100 may analyze and/or process the image data received at step S304 via various image processing techniques to automatically identify one or more objects in the ultrasound images. In some embodiments, a combination of these two approaches may be used, such as wherein the computing device 100 automatically identifies one or more objects in the ultrasound images, and the clinician provides input confirming or rejecting the automatically identified objects. The objects may be the target to be treated during the microwave ablation procedure, other treatment locations, and/or other structures, such as critical structures (e.g. major blood vessels, organs, etc.), which the clinician would like to identify within the EM field.

After identifying the object, the computing device 100 may perform additional albeit optional steps based on the identified object, as described below with reference to optional steps S310-S314. Those skilled in the art will recognize that, in some embodiments, steps S310-S314 may be omitted and that the method 300 may proceed directly from step S308 to steps S316 and S334.

At step S310, the computing device 100 determines a position within the EM field of the one or more objects identified at step S308. In embodiments, the computing device 100 may determine the position of the identified objects based on the position of the ultrasound images within the EM field determined at step S306. For example, once the computing device 100 determines the position of the ultrasound images within the EM field, the computing device 100 may further determine the position of the object identified at step S308 based on the object's position within the ultrasound images. The computing device 100 then, at step S312, marks the position of the object within the EM field. For example, the computing device 100 may store the position of the object within the EM field as determined at step S310. That is, the computing device 100 may store the three-dimensional coordinates of the object within the EM field (e.g. relative to the EM field generator 122) such that the computing device 100 is able to determine a relative distance between the object and the EM sensors 132, 142, 162, as described further below.

The computing device 100 may further, at step S314, cause the display 110 and/or the display 206 to display a virtual marker, such as a graphical marker, corresponding to the stored position of the object. In embodiments, the virtual marker is displayed on the ultrasound images when the position of the virtual marker is included in the displayed ultrasound images. In further embodiments, the virtual marker is displayed separately from the ultrasound images, and may be displayed with increased and/or decreased brightness and/or other visual differentiation based on its position relative to the ultrasound images (e.g. dimmer when behind the ultrasound images and brighter when in front of or co-planar with the ultrasound images).

After step S314, the method 300 splits into two branches—a first branch (steps S316-S332) relates to navigation of the RTP 160, and a second branch (steps S334-S354) relates to navigation of the ablation probe 130. For the purpose of clarity, the two branches will be described independently below, but those skilled in the art will recognize that the various steps of the two branches may be performed concurrently without departing from the scope of the present disclosure.

At step S316, the computing device 100 determines a position of the RTP 160 within the EM field. For example, the computing device 100 may determine the position of the RTP 160 based on the EM tracking data of the EM sensor 162 received at step S302. Thereafter, at step S318, the computing device 100 determines whether the position of the RTP 160 determined at step S316 is proximate the position of the object. For example, the computing device 100 may determine whether the position of the EM sensor 162 is within a predetermined distance of the position of the stored virtual marker which indicates the position of the object. If the computing device 100 determines that the RTP 160 is proximate the object (“YES” at step S318), the method 300 proceeds to step S320, where the computing device 100 provides an alert. The alert may include a proximity warning, such as a visual, haptic, or auditory warning provided by the computing device 100, such as via display 110 and/or 206. The alert may further include a distance and/or direction indicator to indicate a determined distance between the RTP 160 and the object, and a direction from the tip of the RTP 160 to the object.

Thereafter, or if the computing device 100 determines that the RTP 160 is not proximate the object (“NO” at step S318), the method 300 proceeds to step S322, where the computing device 100 determines a trajectory of the RTP 160. In embodiments, the computing device 100 determines the trajectory of the RTP 160 based on the EM tracking data of the EM sensor 162 received at step S302 and/or the position of the RTP 160 determined at step S316. For example, the computing device 100 may determine the trajectory of the RTP 160 based on the pose of the RTP 160 within the EM field. The EM tracking data of the EM sensor 162 may indicate that the RTP 160 is pointed in a particular direction. The computing device 100 may further use data regarding dimensions of the RTP 160, such as a length, width, and/or shape of the RTP 160, to determine the trajectory of the RTP 160. That is, the trajectory of the RTP 160 may indicate the direction in which the RTP 160 is being navigated as well as a maximum distance the RTP 160 may be navigated based on the length of the RTP 160.

Turning now to FIG. 3B, at step S324, the computing device 100 determines whether the trajectory of the RTP 160 intersects with and/or brings the RTP 160 proximate the object. For example, the computing device 100 may determine whether any portion of the trajectory of the RTP 160, as determined at step S322, is within a predetermined distance of the position of the object, as determined at step S310. The predetermined distance may be, for example, 1 mm, 0.5 mm, 0.2 mm, and/or any other distance configurable by the clinician. By determining and displaying the position of the RTP 160 relative to the object, and the distance and direction between the RTP 160 and the object, the computing device 100 provides the clinician with data that may be used to steer the RTP 160 either towards or away from the object. For example, if the object is a critical structure and the purpose of the RTP 160 is to monitor temperature proximate the critical structure to avert damage to the critical structure, the clinician may use the data regarding the position and/or trajectory of the RTP 160 relative to the object to place the RTP 160 proximate the object, but at a safe distance from the object so as not to puncture or otherwise damage the object. Alternatively, if the object is a position where the clinician wishes to place the RTP 160, for example, a position that is a predetermined distance away from the target and from where the progress of the microwave ablation procedure can be monitored, the clinician may use the data regarding the position and/or trajectory of the RTP 160 relative to the object to place the RTP 160 at the desired position—in such a case, the clinician may steer the RTP 160 to the object, as opposed to steering it proximate the object, as described in the previous example.

If the computing device 100 determines that the trajectory brings the RTP 160 proximate the object (“YES” at step S324), the method 300 proceeds to step S326, where the computing device 100 provides an alert. As with the alert provided at step S320, the alert may include a proximity warning, such as a visual, haptic, or auditory warning provided by the computing device 100, such as via display 110 and/or 206. The alert may further include a distance and/or direction indicator to indicate a determined distance between the trajectory of RTP 160 and the object (for example, the portion of the trajectory of RTP 160 that brings the RTP 160 closest to the object), and a direction from the trajectory of the RTP 160 to the object.

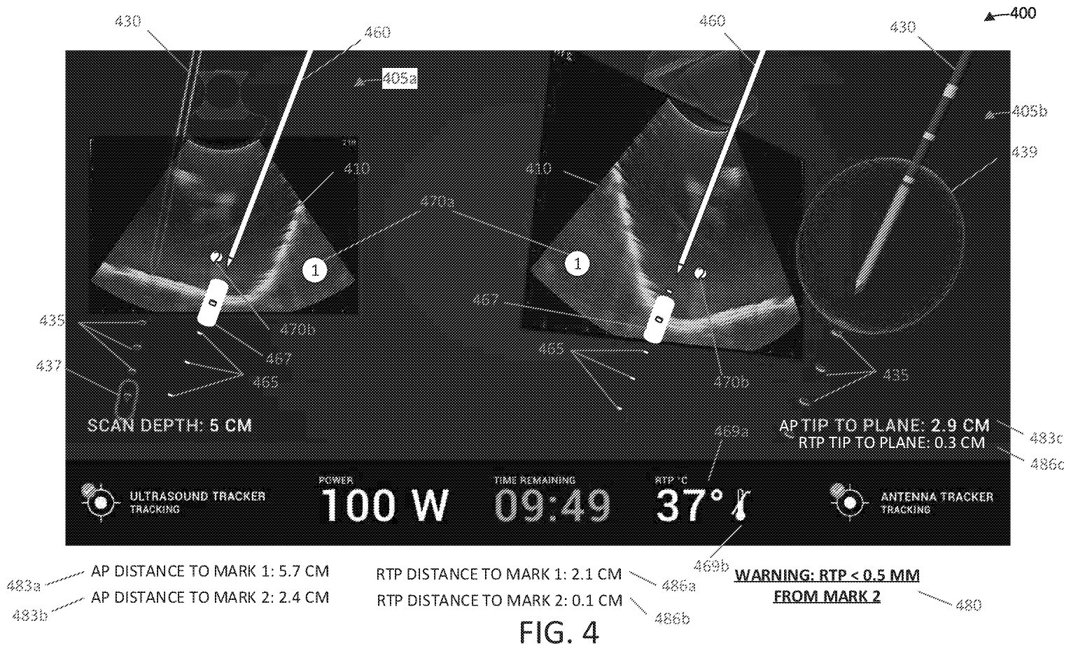

Thereafter, or if the computing device 100 determines that the trajectory of the RTP 160 is not proximate (e.g. not within the predetermined distance of) the object (“NO” at step S324), the method 300 proceeds to step S328, where the computing device 100 determines a point of intersection between the RTP 160 and the ultrasound image plane. Alternatively, if the RTP 160 does not intersect the ultrasound image plane, the computing device 100 determines a point of intersection between the trajectory of the RTP 160 and the ultrasound image plane. In embodiments, the computing device 100 may determine the point of intersection between the RTP 160, or the trajectory of the RTP 160, and the ultrasound image plane based on data regarding the dimensions of the RTP 160, the position of the RTP 160 (determined at step S316), the trajectory of the RTP 160 (determined at step S322), and the position of the ultrasound image plane (determined at step S306). For example, the computing device 100 may determine the point of intersection between the RTP 160, or the trajectory of the RTP 160, by determining whether the data regarding the dimensions of the RTP 160 combined with the position of the RTP 160 and/or the trajectory of the RTP 160 intersect with the position of the ultrasound image plane. The computing device 100 then, at step S330, displays the position of the RTP 160 (as determined at step S316), the trajectory of the RTP 160 (as determined at step S322), and the point of intersection with the ultrasound image plane (as determined at step S328). In embodiments, the computing device 100 may cause the display 110 and/or the display 206 to display the position and trajectory of the RTP 160 and the point of intersection with the ultrasound image plane on the ultrasound images. The position and trajectory of the RTP 160 and the point of intersection with the ultrasound image plane may also be displayed as part of a 3D rendering of the EM field, as shown in FIG. 4 and described further below.

Turning now to FIG. 3C, at step S332, the computing device 100 determines whether the position of the RTP 160 has changed. For example, the computing device 100 may receive additional EM tracking data regarding the EM sensor 162 from the EM tracking system, and may determine based on the additional EM tracking data whether the position of the RTP 160 has changed from the determination at step S316. If the computing device 100 determines that the position of the RTP 160 has changed (“YES” at step S332), the method 300 returns to step S316 where the computing device 100 again determines and/or updates the position of the RTP 160. Alternatively, if the computing device 100 determines that the position of the RTP 160 has not changed (“NO” at step S332), the method 300 proceeds to step S356.

At step S356, the two branches of the method 300 recombine. As such, for purpose of clarity, the second branch of the method 300 (e.g. steps S334-S354) will now be described before continuing with step S356.

Returning now to FIG. 3A, at step S334, the computing device 100 receives configuration settings regarding the ablation probe 130 and/or the microwave ablation procedure. As noted above, in embodiments where a planning and/or configuration phase is performed prior to the start of the method 300, some or all of the configuration settings may be received during such a planning and/or configuration phase. Alternatively, if such a planning and/or configuration phase is not performed prior to the start of the method 300, the configuration settings may be received at step S334. The configuration settings may include a power, temperature, and/or duration of the microwave ablation procedure, as well as a size and/or shape of an ablation zone (e.g. the portion of tissue that will be treated during the microwave ablation procedure).

Thereafter, at step S336, the computing device 100 determines a position of the ablation prove 130 within the EM field. For example, the computing device 100 may determine the position of the ablation probe 130 based on the EM tracking data of the EM sensor 132 received at step S302. The computing device 100 may then, at step S338, determine whether the position of the ablation probe 130, as determined at step S336, is proximate the position of the object, as determined at step S310. For example, the computing device 100 may determine whether the position of the EM sensor 132 is within a predetermined distance of the position of the stored virtual marker which indicates the position of the object. If the computing device 100 determines that the ablation probe 130 is proximate the object (“YES” at step S338), the method 300 proceeds to step S340, where the computing device 100 provides an alert. The alert may include a proximity warning, such as a visual, haptic, or auditory warning provided by the computing device 100, such as via display 110 and/or 206. The alert may further include a distance and/or direction indicator to indicate a determined distance between the ablation probe 130 and the object, and a direction from the tip of the ablation probe 130 to the object.

Next, or if the computing device 100 determines that the ablation probe 130 is not proximate the object (“NO” at step S338), the method 300 proceeds to step S342, where the computing device 100 determines a trajectory of the ablation probe 130. In embodiments, the computing device 100 determines the trajectory of the ablation probe 130 based on the EM tracking data of the EM sensor 132 received at step S302 and/or the position of the ablation probe 130 determined at step S336. For example, the computing device 100 may determine the trajectory of the ablation probe 130 based on the pose of the ablation probe 130 within the EM field. The EM tracking data of the EM sensor 132 may indicate that the ablation probe is pointed in a particular direction. The computing device 100 may further use data regarding dimensions of the ablation probe 130, such as a length, width, and/or shape of the ablation probe 130, to determine the trajectory of the ablation probe 130. That is, the trajectory of the ablation probe 130 may indicate the direction in which the ablation probe 130 is being navigated as well as a maximum distance the ablation probe 130 may be navigated based on the length of the ablation probe 130.

Turning again to FIG. 3B, at step S344, the computing device 100 determines whether the trajectory of the ablation probe 130 intersects with and/or brings the ablation probe 130 proximate the object. For example, the computing device 100 may determine whether any portion of the trajectory of the ablation probe 130, as determined at step S342, is within a predetermined distance of the position of the object, as determined at step S310. The predetermined distance may be, for example, 1 mm, 0.5 mm, 0.2 mm, and/or any other distance configurable by the clinician. By determining and displaying the position of the ablation probe 130 relative to the object, and the distance and direction between the ablation probe 130 and the object, the computing device 100 provides the clinician with data that may be used to steer the ablation probe either towards or away from the object. For example, if the object is a critical structure, the clinician may use the data regarding the position and/or trajectory of the ablation probe 130 relative to the object to ensure the ablation probe 130 is navigated a safe distance from the object so as not to puncture or otherwise damage the object. Alternatively, if the object is a position where the clinician wishes to place the ablation probe 130, for example, the target location, the clinician may use the data regarding the position and/or trajectory of the ablation probe 130 relative to the object to navigate the ablation probe 130 to the object/target.

If the computing device 100 determines that the trajectory brings the ablation probe 130 proximate the object (“YES” at step S344), the method 300 proceeds to step S346, where the computing device 100 provides an alert. As with the alert provided at step S340, the alert may include a proximity warning, such as a visual, haptic, or auditory warning provided by the computing device 100, such as via display 110 and/or 206. The alert may further include a distance and/or direction indicator to indicate a determined distance between the trajectory of ablation probe 130 and the object (for example, the portion of the trajectory of ablation probe 130 that brings the ablation probe 130 closest to the object), and a direction from the trajectory of the ablation probe to the object.

Thereafter, or if the computing device 100 determines that the trajectory of the ablation probe 130 is not proximate (e.g. not within the predetermined distance of) the object (“NO” at step S344), the computing device 100 determines a position of a projected ablation zone. In embodiments, the computing device 100 determines the position of the projected ablation zone based on the position of the ablation probe 130, as determined at step S336, and the configuration settings received at step S334. In particular, the computing device 100 may determine the position of the distal radiating portion 134 of the ablation probe 130, which is a predetermined distance from the EM sensor 132, and determine the size and shape of the ablation zone that will be created based on the power, temperature, and/or duration settings received at step S334.

Then, at step S350, the computing device 100 determines a point of intersection between the ablation probe 130 and the ultrasound image plane. Alternatively, if the ablation probe 130 does not intersect the ultrasound image plane, the computing device 100 determines a point of intersection between the trajectory of the ablation probe 130 and the ultrasound image plane. In embodiments, the computing device 100 may determine the point of intersection between the ablation probe 130, or the trajectory of the ablation probe 130, and the ultrasound image plane based on data regarding the dimensions of the ablation probe 130, the position of the ablation probe 130 (determined at step S336), the trajectory of the ablation probe 130 (determined at step S342), and the position of the ultrasound image plane (determined at step S306). For example, the computing device 100 may determine the point of intersection between the ablation probe 130, or the trajectory of the ablation probe 130, by determining whether the data regarding the dimensions of the ablation probe 130 combined with the position of the ablation probe 130 and/or the trajectory of the ablation probe 130 intersect with the position of the ultrasound image plane.

The computing device 100 then, at step S352, displays the position of the ablation probe 130 (as determined at step S336), the trajectory of the ablation probe 130 (as determined at step S342), and the point of intersection with the ultrasound image plane (as determined at step S350). The computing device 100 may further display the projected ablation zone, as determined at step S348. In embodiments, the computing device 100 may cause the display 110 and/or the display 206 to display the position and trajectory of the ablation probe 130 and the point of intersection with the ultrasound image plane, as well as the projected ablation zone, on the ultrasound images. The position and trajectory of the ablation probe 130 and the point of intersection with the ultrasound image plane, as well as the projected ablation zone, may also be displayed as part of a 3D rendering of the EM field, as shown in FIG. 4 and described further below.

Turning again to FIG. 3C, at step S354, the computing device 100 determines whether the position of the ablation probe 130 has changed. For example, the computing device 100 may receive additional EM tracking data regarding the EM sensor 132 from the EM tracking system, and may determine based on the additional EM tracking data whether the position of the ablation probe 130 has changed from the determination at step S336. If the computing device 100 determines that the position of the ablation probe 130 has changed (“YES” at step S354), the method 300 returns to step S336 where the computing device 100 again determines and/or updates the position of the ablation probe 130. Alternatively, if the computing device 100 determines that the position of the ablation probe 130 has not changed (“NO” at step S354), the method 300 proceeds to step S356.

At step S356, the computing device 100 determines whether the ablation probe 130 has been activated. In embodiments, the computing device 100 determines that the ablation probe 130 has been activated by receiving a signal from the ablation probe 130 and/or from the generator 115 indicating that microwave energy is being provided to the ablation probe 130 and is being emitted by the distal radiating portion 134 of the ablation probe 130. Once the ablation probe 130 is activated, the computing device 100 determines that the navigation phase for the current target has been completed and a treatment phase has commenced. If the computing device 100 determines that the ablation probe 130 has not been activated (“NO” at step S356), the method 300 returns to steps S332 and/or S354, where the computing device 100 again determines whether the position of the RTP 160 and/or the ablation probe 130 has changed.

Alternatively, if the computing device 100 determines that the ablation probe 130 has been activated (“YES” at step S356), the method 300 proceeds to step S358, where the computing device 100 receives a temperature measurement from the RTP 160. The computing device 100 then, at step S360, displays the temperature measurement. In embodiments, the computing device 100 may cause the display 110 and/or the display 206 to display the temperature measurement. Thereafter, or concurrently therewith, at step S362, the computing device 100 determines whether the temperature measurement received at step S358 is greater than a first threshold temperature. In embodiments where the RTP 160 is used to monitor temperature at a critical structure to prevent damage to the critical structure, the first threshold temperature may be a predetermined temperature that is a safe temperature up to which the critical structure can be heated without causing damage to the critical structure. In embodiments where the RTP 160 is used to monitor the progress of the microwave ablation procedure, the first threshold temperature may be a temperature indicating a particular progress level of the microwave ablation procedure. In such cases, the first threshold temperature may either be predetermined and/or may be automatically determined by the computing device 100 based on the distance between the RTP 160 and the ablation probe 130, particularly the distal radiating portion 134, and may be correlated based on the temperature that tissue at the distance of the RTP 160 from the distal radiating portion 134 should be heated at the particular progress level of the microwave ablation procedure. If the computing device 100 determines that the temperature measurement received at step S358 is greater than the first threshold temperature (“YES” at step S362), the method 300 proceeds to step S364. Alternatively, if the computing device 100 determines that the temperature measurement received at step S358 is not greater than the first threshold temperature (“NO” at step S362), the method 300 proceeds to step S378.

Turning now to FIG. 3D, at step S364, the computing device 100 causes the generator 115 and/or the ablation probe 130 to stop the microwave ablation procedure. In embodiments, the computing device 100 may send a signal to the generator 115 to stop providing microwave energy to the ablation probe 130. Additionally or alternatively, the computing device 100 may send a signal to the ablation probe 130 to stop emitting microwave energy. Thereafter or concurrently therewith, at step S366, the computing device 100 displays an indicator that the temperature measurement received at step S358 is above the first threshold temperature. In embodiments, the computing device 100 may cause the display 110 and/or the display 206 to display an indicator that the temperature measurement is above the first threshold temperature. The computing device 100 then, at step S368, determines whether the microwave ablation treatment phase for the current target is complete. The determination may be based on the configuration settings received at step S334 and/or user input received from the clinician. For example, if the duration and/or temperature of the ablation treatment for the current target matches the duration and/or temperature value received in the configuration settings, the computing device 100 may determine that the treatment phase for the current target is complete. If the computing device 100 determines that the treatment phase for the current target is complete (“YES” at step S368), the method 300 proceeds to step S386. Alternatively, if the computing device 100 determines that the treatment phase for the current target is not complete (“NO” at step S368), the method 300 proceeds to step S370.

At step S370, the computing device 100 receives a new temperature measurement from the RTP 160. The computing device 100 then, at step S372, determines whether the new temperature measurement is below a second threshold temperature. In embodiments where the RTP 160 is used to monitor temperature at a critical structure to prevent damage to the critical structure, the second threshold temperature may be a predetermined temperature that is less than the first threshold temperature by a sufficient amount to allow the tissue surrounding the critical structure to cool and avoid damage to the critical structure. In embodiments where the RTP 160 is used to monitor the progress of the microwave ablation procedure, the second threshold temperature may be a temperature that is less than the first threshold temperature by a predetermined amount sufficient to allow the tissue surrounding the RTP 160 to cool before the microwave ablation procedure continues. In such cases, the second threshold temperature may either be predetermined and/or may be automatically determined by the computing device 100 based on the distance between the RTP 160 and the ablation probe 130, particularly the distal radiating portion 134. Further, in embodiments where the ablation treatment involves both heating via microwave ablation and active cooling via cryo or another active cooling method, the second temperature threshold may be a low temperature threshold indicating that the tissue has been sufficiently cooled before again being heated. If the computing device 100 determines that the temperature measurement received at step S370 is not below the second threshold temperature (“NO” at step S372), the method 300 returns to step S370, where another new temperature measurement is received, and this cycle may repeat until the received temperature measurement indicates that the temperature has dropped below the second temperature threshold. Alternatively, if the computing device 100 determines that the temperature measurement received at step S370 is below the threshold temperature (“YES” at step S372), the method 300 proceeds to step S374.

Thereafter or concurrently therewith, at step S374, the computing device 100 displays an indicator that the temperature measurement received at step S370 is below the second threshold temperature. In embodiments, the computing device 100 may cause the display 110 and/or the display 206 to display an indicator that the temperature measurement is below the second threshold temperature. In some embodiments, a single temperature indicator may be used, and thus the temperature indicator displayed at step S366 may be updated at step S374 to indicate that the temperature is no longer above the first threshold. The computing device 100 then, at step S376, causes the generator 115 and/or the ablation probe 130 to resume the microwave ablation procedure. In embodiments, the computing device 100 may send a signal to the generator 115 to resume providing microwave energy to the ablation probe 130. Additionally or alternatively, the computing device 100 may send a signal to the ablation probe 130 to resume emitting microwave energy. Thereafter, the method 300 returns to step S358 where a new temperature measurement is received.

Turning now to FIG. 3E, at step S378, the computing device 100 determines a progress of the microwave ablation procedure. In embodiments, the computing device 100 may determine the progress of the microwave ablation procedure based on the temperature measurement received at step S358. For example, the computing device 100 may determine, based on the distance between the RTP 160 and the ablation probe 130, particularly the distal radiating portion 134, whether the temperature measurement received at step S358 indicates that the microwave ablation procedure has progressed to one or more predetermined levels. The levels may be based on temperature thresholds and may be correlated to the distance between the RTP 160 and the ablation probe 130.

Thereafter, at step S380, the computing device 100 determines whether the microwave ablation treatment phase for the current target is complete. For example, the computing device 100 may determine whether the progress of the microwave ablation procedure determined at step S378 indicates that the treatment phase for the current target is complete. If the computing device 100 determines that the treatment phase for the current target is not complete, the method 300 returns to step S358, where a new temperature measurement is received. Alternatively, if the computing device 100 determines that the treatment phase for the current target has been completed, the method 300 proceeds to step S382, where the computing device 100 causes the generator 115 and/or the ablation probe 130 to stop the microwave ablation procedure. In embodiments, the computing device 100 may send a signal to the generator 115 to stop providing microwave energy to the ablation probe 130. Additionally or alternatively, the computing device 100 may send a signal to the ablation probe 130 to stop emitting microwave energy.

Thereafter, at step S384, the computing device 100 displays an indication that the microwave ablation treatment phase for the current target is complete. In embodiments, the computing device 100 may cause the display 110 and/or the display 206 to display an indication that the treatment phase for the current target is complete. Next, at step S386, the computing device 100 determines whether there are additional targets to be treated. In embodiments, the computing device 100 may determine whether there are additional targets to be treated based on the configuration settings received at step S334 and/or based on user input received from the clinician. If the computing device 100 determines that there are one or more additional targets to be treated (“YES” at step S386), the method 300 returns to step S354, where the computing device 100 again determines whether the position of the ablation probe 130 has changed. Alternatively, if the computing device 100 determines that there are no additional targets to be treated (“NO” at step S386), the method 300 ends.

Turning now to FIG. 4, there is shown an illustrative GUI 400 which may be displayed by the computing device 100 during the microwave ablation procedure. The GUI 400 includes two views 405a, 405b. The view 405a shows an ultrasound image 410 normal to the ultrasound image plane with indicators of a virtual ablation probe 430 and a virtual RTP 460 shown based on the positions of the ablation probe 130 and the RTP 160 relative to the ultrasound sensor 140. The view 405a further shows a first virtual marker 470a and a second virtual marker 470b. The marker 470 may correspond to a marked position of a target, while the marker 470b may correspond to a marked position of a critical structure. Also shown are rings 435, 465 corresponding to the trajectories of the virtual ablation probe 430 and the virtual RTP 460, respectively. Additionally, the point of intersection between the virtual ablation probe 430 and the ultrasound image plane is shown by an obround shape 437, which indicates the point of intersection between the virtual ablation probe 430 and a front and back (not expressly shown in FIG. 4) of the ultrasound image plane, accounting for a predetermined margin of error. Similarly, the point of intersection between the virtual RTP 460 and the ultrasound image plane is shown by an obround shape 467, which indicates the point of intersection between the virtual RTP 460 and a front and back (not expressly shown in FIG. 4) of the ultrasound image plane, accounting for a predetermined margin of error.

The view 405b shows all the same elements described above that are shown in the view 405a. However, the view 405b differs from the view 405a in that the view 405b is not shown normal to the ultrasound image plane, but rather fixed based on the EM field. That is, the view 405b shows the various elements (e.g. the ultrasound image 410, the virtual ablation probe 430, and the virtual RTP 460, among others) based on their respective positions within the EM field. That means, for example, that the ultrasound image 410 will move about the view 405b based on the position of the ultrasound sensor 140 within the EM field. Similarly, the virtual ablation probe 430 and the virtual RTP 460, along with their respective trajectory rings 435, 465 and obrounds 437, 467 will move about the view 405b based on the position of the ablation probe 130 and the RTP 160 within the EM field. A notable difference is the way the virtual markers 470a, 470b are displayed in the view 405a compared to the view 405b. In the view 405a, because the view 405a is displayed normal to the ultrasound image plane, the virtual markers 470a, 470b will move based on the position of the ultrasound sensor 140 within the EM field. However, in the view 405b, since the view 405b is fixed relative to the EM field, and the markers 470a, 470b correspond to particular positions marked within the EM field, the markers 470a, 470b will remain in a fixed position in the view 405b.

Also shown in the GUI 400 is a temperature indicator 469a corresponding to the current temperature measurement received from the RTP 160, and a threshold indicator 469b corresponding to a determination whether the temperature measurement received from the RTP 160 is above a threshold. In embodiments, the threshold indicator 469b may change based on the determination whether the temperature measurement received from the RTP 160 is above or below various thresholds. Additionally, various distance indicators may also be displayed as part of the GUI 400. For example, the GUI 400 may include indicators 483a, 483b, 483c corresponding to a distance between the ablation probe 130 and the first marker 470a, a distance between the ablation probe 130 and the second marker 470b, and a distance between the ablation probe 130 and the ultrasound image plane, respectively. Likewise, the GUI may include indicators 486a, 486b, 486c corresponding to a distance between the RTP 160 and the first marker 470a, a distance between the RTP 160 and the second marker 470b, and a distance between the RTP 160 and the ultrasound image plane, respectively. The GUI 400 may also include a warning view 480 where proximity warnings or other alerts may be displayed.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. An electrosurgical system comprising:
a temperature probe including a temperature sensor;
an ultrasound transducer;
an electromagnetic (EM) tracking system including:
an EM field generator, and
an EM sensor coupled to the temperature probe,
a display device; and
a computing device including a processor and a memory storing instructions which, when executed by the processor, cause the computing device to:
receive, from the EM tracking system, tracking data indicating a position of the EM sensor,
receive, from the ultrasound transducer, ultrasound images,
determine a position of the temperature probe based on the position of the EM sensor,
determine a trajectory of the temperature probe based on the position of the EM sensor, and
cause the display device to display an indication of the position and the trajectory of the temperature probe on the ultrasound images.

2. The system according to claim 1, further comprising:
a microwave energy source; and
a microwave ablation antenna operably coupled to the microwave energy source,
wherein the EM tracking system further includes a second EM sensor coupled to the microwave ablation antenna.

3. The system according to claim 2, wherein the instructions, when executed by the processor, further cause the computing device to:
receive, from the EM tracking system, tracking data indicating a position of the second EM sensor,
determine a position of the microwave ablation antenna based on the position of the second EM sensor,
determine a trajectory of the microwave ablation antenna based on the position of the second EM sensor, and
cause the display device to display an indication of the position and the trajectory of the microwave ablation antenna on the ultrasound images.

4. The system according to claim 3, wherein the instructions, when executed by the processor, further cause the computing device to:
receive ablation procedure configuration settings;
determine a projected ablation zone based on the ablation procedure configuration settings;
determine a position of the projected ablation zone based on the position of the microwave ablation antenna; and
cause the display device to display an indication of the position of the projected ablation zone on the ultrasound images.

5. The system according to claim 2, wherein the instructions, when executed by the processor, further cause the computing device to:
receive a temperature measurement from the temperature probe; and
determine whether the temperature measurement is above a threshold.

6. The system according to claim 5, wherein the instructions, when executed by the processor, further cause the computing device to, when it is determined that the temperature measurement is above the threshold, cause the microwave energy source to stop providing microwave energy to the microwave ablation antenna.

7. The system according to claim 5, wherein the instructions, when executed by the processor, further cause the computing device to, when it is determined that the temperature measurement is above the threshold, cause the display device to display an indication that the temperature measurement is above the threshold.

8. The system according to claim 5, wherein the instructions, when executed by the processor, further cause the computing device to:
receive ablation procedure configuration settings,
wherein the threshold is determined based on the ablation procedure configuration settings.

9. The system according to claim 5, wherein the threshold is a predetermined value.

10. The system according to claim 1, wherein the instructions, when executed by the processor, further cause the computing device to:
receive a temperature measurement from the temperature probe;
determine a progress of an ablation procedure based on the temperature measurement.

11. The system according to claim 10, wherein the instructions, when executed by the processor, further cause the computing device to cause the display device to display an indication of the progress of the ablation procedure.

12. The system according to claim 10, wherein the instructions, when executed by the processor, further cause the computing device to:
determine, based on the progress of the ablation procedure, whether the ablation procedure has been completed.

13. The system according to claim 12, wherein the instructions, when executed by the processor, further cause the computing device to, when it is determined that the ablation procedure has been completed, cause the microwave energy source to stop providing microwave energy to the microwave ablation antenna.

14. The system according to claim 12, wherein the instructions, when executed by the processor, further cause the computing device to, when it is determined that the ablation procedure has been completed, cause the display device to display an indication that the ablation procedure has been completed.

15. The system according to claim 1, wherein the instructions, when executed by the processor, further cause the computing device to:
receive a temperature measurement from the temperature probe; and
cause the display device to display an indication of the temperature measurement.

16. The system according to claim 1, wherein the EM tracking system further includes a second EM sensor coupled to the ultrasound transducer, and wherein the instructions, when executed by the processor, further cause the computing device to:
determine a position of an ultrasound image plane generated by the ultrasound transducer based on the position of the second EM sensor;
determine a point of intersection between the trajectory of the temperature probe and the ultrasound image plane; and
cause the display device to display an indication of the point of intersection between the trajectory of the temperature probe and the ultrasound image plane.

17. The system according to claim 1, wherein the EM tracking system further includes a second EM sensor coupled to the ultrasound transducer, and wherein the instructions, when executed by the processor, further cause the computing device to:
determine a position of an ultrasound image plane generated by the ultrasound transducer, based on the position of the second EM sensor;
determine a point of intersection between the temperature probe and the ultrasound image plane; and
cause the display device to display an indication of the point of intersection between the temperature probe and the ultrasound image plane.

18. The system according to claim 1, wherein the tracking data is first tracking data and the position of the EM sensor is a first position of the EM sensor, and wherein the instructions, when executed by the processor, further cause the computing device to:
receive, from the EM tracking system, second tracking data indicating a second position of the EM sensor,
determine an updated position of the temperature probe based on the second position of the EM sensor,
determine an updated trajectory of the temperature probe based on the second position of the EM sensor, and
cause the display device to display an indication of the updated position and the updated trajectory of the temperature probe on the ultrasound images.

19. A method comprising:
receiving tracking data indicating a position of an EM sensor coupled to a temperature probe;
receiving, from an ultrasound transducer, ultrasound images;
determining a position of the temperature probe based on the position of the EM sensor;
determining a trajectory of the temperature probe based on the position of the EM sensor; and
causing a display device to display an indication of the position and the traj ectory of the temperature probe on the ultrasound images.

20. A non-transitory computer-readable medium storing instructions which, when executed by a processor, cause a computing device to:
receive tracking data indicating a position of an EM sensor coupled to a temperature probe,
receive, from an ultrasound transducer, ultrasound images,
determine a position of the temperature probe based on the position of the EM sensor,
determine a trajectory of the temperature probe based on the position of the EM sensor, and
cause a display device to display an indication of the position and the trajectory of the temperature probe on the ultrasound images.
